(19) United States
(12) Patent Application Publication
(10) Pub. No.: US 2026/0301897 A1
(43) Pub. Date: Oct. 1, 2026

(54) SYSTEMS AND METHODS TO ASSOCIATE MEDICAL DEVICE DATA WITH PATIENT DATA

(71) Applicant: Covidien LP, Mansfield, MA (US)

(72) Inventors: David A. Fox, Longmont, CO (US); Alok Yadav, Lafayette, CO (US); Praveen Bonam, Hyderabad (IN); Sangeeta Rao Chaganti, Hyderabad (IN)

(21) Appl. No.: 19/578,445

(22) Filed: Mar. 25, 2026

Related U.S. Application Data
(60) Provisional application No. 63/780,732, filed on Mar. 31, 2025.

(51) Int. Cl. G16H 10/60; G16H 40/67

(52) U.S. Cl. CPC G16H 10/60; G16H 40/67

(57) ABSTRACT

The systems and methods disclosed herein permit a caregiver to associate parameters from a device (e.g., medical device) via an intervening user device, such as a mobile device or a tablet, operated by a caregiver. For example, the disclosed techniques permit the caregiver to associate the parameters from the device with patient information stored in an electronic medical record (EMR) system, validate the parameters, and store the validated parameters in the EMR system. The parameters may include physiological parameters of the patient, settings of the device (e.g., settings), outputs (e.g., video data and/or image data) of the device, or any combination thereof. The parameters may be stored with an indication of validation, which may include a clinician identifier, a point in time of the validation, and so on. Storing validated data in the EMR system may improve patient care by providing accurate and/or reliable physiological parameters.

### CROSS-REFERENCE TO RELATED APPLICATIONS

This application claims priority to and the benefit of U.S. Application Ser. No. 63/780,732, filed Mar. 31, 2025, the entire disclosure of which is hereby incorporated by reference.

### TECHNICAL FIELD

The present disclosure generally relates to patient monitoring, and more particularly, to systems and methods for associating parameters from a device with patient information stored in an electronic medical record (EMR) system, validating the parameters, and storing the validated parameters in electronic medical record (EMR) systems.

### BACKGROUND

Medical devices are widespread in a patient care environment. Data generated from these medical devices may be added to the patient's electronic medical records (e.g., electronic health records (EHRs)) that are stored in a database (e.g., electronic medical record (EMR) system). In certain cases, the EMR system interface may directly receive data from medical devices that are electronically associated with a particular patient via the patient's identification information. However, certain medical devices may not support this electronic association, and the EMR system may also be configured to accept manual data entry.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a system may include a medical device to monitor a parameter of a patient or administer a treatment to the patient, the medical device including a device identifier and a validation system communicatively coupled to the medical device. The validation system may include a processor and a memory storing instructions that, when executed by the processor, cause the processor to perform operations to cause a mobile device to receive the device identifier and a patient identifier, display the device identifier and the patient identifier on a graphical user interface (GUI) displayed on the mobile device, and receive user input associating the medical device with the patient based on user input from the GUI, wherein the patient is associated with the patient identifier. The processor may also perform operations to send a validated association based on the user input and instruct the mobile device to display the parameter responsive to receiving the validation.

In another embodiment, a system may include a medical device to monitor a first parameter of a patient or a second parameter of the medical device, the medical device including a device identifier, a mobile device to acquire a patient identifier and the device identifier, and a validation system coupled to the medical device and the mobile device. The validation system may include a processor and a memory storing instructions that, when executed by the processor, cause the processor to receive the first parameter or the second parameter from the medical device, instruct the mobile device to display the first parameter or the second parameter, receive a first user input indicative of validating the first parameter or the second parameter from the mobile device at a point in time, and transmit the validated first parameter or the validated second parameter to an electronic medical record system with an indication of validation.

In another embodiment, a system may include a medical device to monitor a patient or administer a treatment to the patient, wherein the medical device outputs a parameter indicative of a physiological parameter of the patient, a validation system communicatively coupled to the medical device and an electronic medical record system, the validation system receives the parameter and transmits the parameter to the electronic medical record system, and a mobile device communicatively coupled to the validation system, the mobile device operating a software application programmed with instructions to activate a camera, acquire, via the camera, a device identifier of the medical device, and receive device information associated with the medical device in response to transmitting the device identifier to the validation system. The mobile device may also acquire, via the camera, a patient identifier of the patient, receive patient information associated with the patient in response to transmitting the patient identifier to the validation system, validate the device information and the patient information in response to receiving a first user selection from the GUI, validate the parameter in response to receiving a second user selection from the GUI displaying the parameter, and display a confirmation of transmitting the parameter to the electronic medical record system with an indication of validation.

Various refinements and features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a block diagram of a validation environment, in accordance with an aspect of the present disclosure;

FIG. 2 is a schematic illustration of a workflow of validating physiological parameters within the validation environment of FIG. 1, in accordance with an aspect of the present disclosure;

FIG. 3 is a schematic illustration of an example graphical user interface (GUI) displayed by a mobile device prior to receiving the device identifier and a patient identifier within the validation environment of FIG. 1, in accordance with an aspect of the present disclosure;

FIG. 4 is a schematic illustration of an example GUI displayed by the mobile device within the validation environment of FIG. 1, where the GUI includes information associated with the device identifier and/or the patient identifier, in accordance with an aspect of the present disclosure;

FIG. 5 is a schematic illustration of an example GUI displayed by the mobile device of the physiological parameter and a GUI displayed by the medical device within the validation environment of FIG. 1, in accordance with an aspect of the present disclosure;

FIG. 6 is a schematic illustration of an example GUI displayed by the mobile device within the validation environment of FIG. 1, where the GUI displays a physiological parameter as a waveform, in accordance with an aspect of the present disclosure;

FIG. 7 is a schematic illustration of an example GUI displayed by the mobile device within the validation environment of FIG. 1, where the GUI displays physiological parameters as values, in accordance with an aspect of the present disclosure;

FIG. 8 is a schematic illustration of an example GUI displayed by the mobile device within the validation environment of FIG. 1, where the GUI displays physiological parameters including a missing value, in accordance with an aspect of the present disclosure;

FIG. 9 is a schematic illustration of an example GUI displayed by the mobile device within the validation environment of FIG. 1, where the GUI displays pending records, in accordance with an aspect of the present disclosure;

FIG. 10 is a flow diagram of an example method for validating physiological parameters using the mobile device in the validation environment of FIG. 1, in accordance with an aspect of the present disclosure; and

FIG. 11 is a flow diagram of an example method for validating and storing the physiological parameter in an electronic medical record (EMR) system in the validation environment of FIG. 1, in accordance with an aspect of the present disclosure.

### DETAILED DESCRIPTION

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Patients may be monitored in a localized environment (e.g., patient room) by medical devices, such as patient medical devices. Additionally or alternatively, patients may be treated and/or imaged in the localized environment and/or an additional localized environment by the medical devices. A clinician may associate physiological parameters of a patient from a medical device with the patient's electronic medical records (e.g., electronic health records) stored in a database (e.g., electronic medical record (EMR) system) using an interface of the medical device itself. In such an example, data communicated to the EMR system by the patient-associated medical device includes patient identification information that permits the EMR system to associate the monitoring data with a particular patient EMR. However, certain patient medical devices may not support associating a particular patient with the medical device via a device interface. In such cases, the clinician can manually associate the medical device data with the correct patient. However, the process for associating the physiological parameters and/or the medical device with the patient's electronic health records may be time-consuming, tedious, and prone to human error. Further, certain EMR systems may not offer a manual association option and, therefore, can only support patient medical devices that are capable of communicating patient-associated data from the device itself.

With the foregoing in mind, the present disclosure generally relates to the field of patient monitoring and/or treatment, and more particularly, to supporting association and validation of parameters from a medical device via an intervening user device, such as a mobile device or tablet, operated by a caregiver. For example, the disclosed techniques permit a caregiver (e.g., clinician, doctor, nurse, technician) to associate parameters from the medical device with patient information in an electronic medical record (EMR) system, validate the parameters (e.g., validate a screenshot of the parameters, video data and/or image taken generated by the medical device, settings of the medical device), and store the validated parameters in the EMR system. As part of association and/or validation, the clinician may interact with a user device that receives unassociated and/or unvalidated parameters from the medical device. Via a user device interface, the clinician can provide inputs to associate and/or validate the parameters displayed on the medical device at a point in time. If the displayed parameters correspond to the parameters displayed on the medical device and to the correct patient identification information, the clinician may validate the parameters. As such, the parameters may be stored in the EMR system with an indication of the validation and/or a time point of the validation.

Storing validated data in the EMR system may improve patient care by providing accurate and/or reliable parameters, streamline workflow of the clinician by reducing or eliminating the step of correcting and/or validating the parameters, and so on. If the parameters displayed on the mobile device do not match the parameters displayed on the medical device and/or are not associated with the correct patient, the clinician may not validate the parameters. For example, the clinician may wait a subsequent period of time for additional parameters to be available for validation. In some cases, the parameters may be communicated from the medical device without validation, which may trigger an indication in the EMR system that the parameters may be less reliable than validated parameters. In another example, the clinician may select to delete the parameters. In other instances, the clinician may view the parameters and determine that the parameters include artifacts, such as distortions to the parameter. For example, an image generated by the medical device may include image artifacts. The clinician may not validate the parameters based on identifying the artifacts. Moreover, the clinician may provide an indication of the artifacts, and the parameters may be stored with the indication, thereby providing an indication that the parameters may be less reliable than validated parameters and/or other parameters stored in the EMR system. Thus, the present techniques improve patient monitoring and/or treatment by streamlining the workflow for associating parameters from a medical device with patient information stored in the EMR system, validating the parameters at a point in time, and providing the validated parameters for storage in the EMR system.

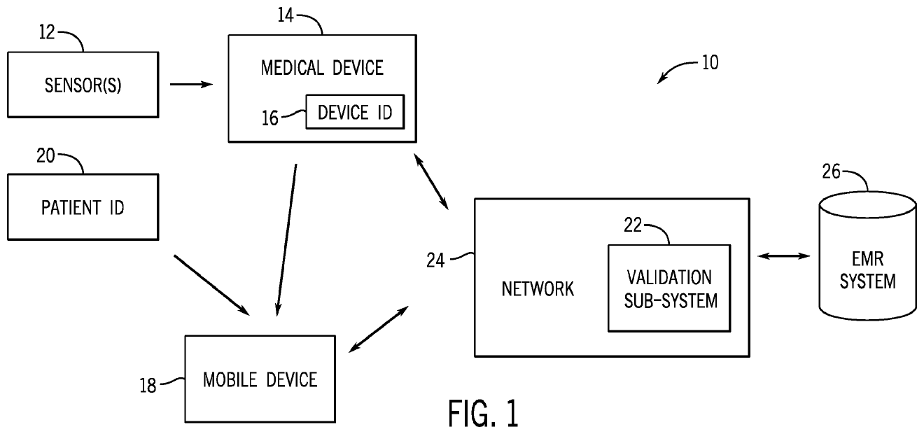

Turning now to FIG. 1, an example validation environment 10 for patient monitoring and/or treatment is illustrated. The validation environment 10 may include sensors 12, a medical device 14, a device identifier (ID) 16, a mobile device 18, a patient ID 20, and/or a validation sub-system 22. It may be appreciated that the validation environment 10 may include more components or fewer components than illustrated.

The validation environment 10 may include a localized environment (e.g., waiting room, triage, patient room) with a patient. For example, a clinician may place one or more sensor(s) 12 to a patient to monitor physiological parameters of the patient. The one or more sensor(s) 12 may include a patch, a band, a wrist-worn device (e.g., watch), and so on. The patient may wear the one or more sensor(s) 12 that individually or collectively monitor physiological parameters of the patient and generate one or more signal(s) indicative of the physiological parameters. For example, the one or more sensor(s) 12 may include blood pressure sensors, heart rate sensors, temperature sensors, oxygen saturation sensors, and so on. The medical device 14 may include any suitable medical device (e.g., bedside medical device, treatment device, imaging device) that generates clinical data that may be validated prior to storage in a patient's electronic medical record. For example, the medical device 14 may include treatment devices, therapeutic devices, and/or imaging devices that capture physiological parameters of the patient and/or provide treatment to the patient. In certain instances, the physiological parameters may also include parameters (e.g., settings) of the medical device 14 used to capture the physiological parameters of the patient and/or administer treatment to the patient. By way of example, the medical device 14 may include a ventilator and the physiological parameters may include ventilator settings used during patient treatment. In another example, the physiological parameters may include video data, image data, and/or audio data generated by the medical device 14 during the patient monitoring and/or treatment session. The medical device 14 may continuously or intermittently monitor physiological parameters of the patient during operation (e.g., patient monitoring session, patient treatment session).

The medical device 14 may be communicatively coupled (e.g., wired or wireless) to the one or more sensor(s) 12 to receive the monitored physiological parameters. In operation, the medical device 14 may calculate or measure one or more physiological parameter(s) of the patient based on one or more signal(s) from the one or more sensor(s) 12. The physiological parameters may include heart rate, respiration rate, blood oxygen level, temperature, and so on. Additionally or alternatively, the medical device 14 may calculate a variety of physiological parameters, such as arterial blood oxygen saturation, regional or tissue oxygen saturation, pulse rate, respiration rate, blood pressure, blood pressure characteristic measure, autoregulation status, brain activity, temperature, or any other suitable physiological parameter. As discussed herein, the physiological parameters may also include device settings, imaging settings, treatment settings, and so on. For example, the physiological parameters may include ventilator settings used to administer a treatment to the patient, imaging settings used to generate video data and/or image data of the patient, and so on.

The medical device 14 may include a user interface with a display to display the physiological parameters of the patient during the patient monitoring and/or treatment session. The medical device 14 may also include a device identifier (ID) 16 that may uniquely identify the medical device 14 and/or physiological parameters monitored by the medical device 14. The device ID 16 may be positioned on an exterior surface of the medical device 14 and may include a machine-readable representation of data. In certain instances, the device ID 16 may be positioned on a component of the medical device 14. For example, the device ID 16 may be positioned on a sensor, a laryngoscope, a tube, a blood pressure cuff, and so on. The device ID 16 may include a barcode or a quick response (QR) code that may store and/or transmit device information of the medical device 14. In other instances, the device ID 16 may include text, a string of letters, a string of numbers, or both that may be associated with the device information. The device information may include a commercial name, a model, a reference number, a serial number, a function, and so of the medical device 14. The medical device 14 may display the device information on the exterior surface of the patient medical device, such that the device information may be visible to the clinician. In certain cases, the device ID 16 may also be included in data communicated from the medical device 14 to other devices in the system.

The clinician may use a mobile device 18 to capture the device ID 16 of the medical device 14. In one example, the mobile device 18 may store a software application that, when executed by the mobile device 18, operates to activate a camera to scan the device ID 16. In another example, the mobile device 18 may activate a radio frequency reader, such as an NFC reader, that can capture radio frequency signals from an RFID tag that stores the device ID 16. The mobile device 18 may display the device information responsive to capture of the device ID 16. The mobile device 18 may be communicatively coupled to a validation sub-system 22 that may store the device information and/or identify the device information in a database. The mobile device 18 may transmit the device ID 16 to the validation sub-system 22 via a network 24 and the validation sub-system 22 may identify and transmit the device information based on the device ID 16. The clinician may compare the device information displayed on the mobile device 18 to the device information on the medical device 14 to validate the medical device 14. For example, if the device information displayed on the mobile device 18 matches the device information on the medical device 14, the clinician may input an indication of validation. If the device information displayed on the mobile device 18 does not match the device information on the medical device 14, the clinician may input an indication of the mismatch. The mobile device 18 may prompt the clinician to capture (e.g., scan) the device ID 16 and repeat the process until an indication of validation is received. In certain instances, the mobile device 18 may prompt the user to input the device ID 16 via a graphical user interface (GUI). For example, the clinician may input a string of letters corresponding to the device ID 16 and the validation sub-system 22 may identify the device information based on the string of letters. In an embodiment, the validation sub-system 22 may include additional components, such as the mobile device 18.

The clinician may use the mobile device 18 to capture a patient ID 20 on a bracelet worn by the patient to receive patient information. The patient ID 20 may include a barcode, a QR code, a string of numbers, a string of letters, or any combination thereof. The mobile device 18 may, via the application, activate the camera to scan the patient ID 20 and display patient information. In another example, the mobile device 18 may activate a radio frequency reader, such as an NFC reader, that can capture radio frequency signals from an RFID tag that stores the patient ID 20. The mobile device 18 may transmit the patient ID 20 to the validation sub-system 22 via a network 24 and the validation sub-system 22 may identify and transmit the patient information based on the patient ID 20. The patient information may include a patient name, a birth date, an address, a gender, a phone number, and so on. If the patient information displayed on the mobile device 18 matches patient information on the bracelet worn by the patient, then the clinician may input an indication of validation. If the patient information displayed on the mobile device 18 does not match the patient information on the bracelet, the clinician may input an indication of mismatch. The mobile device 18 may prompt the clinician to repeat this process until an indication of validation of association of the device information with the patient identification information is received. In certain instances, the mobile device 18 may prompt the clinician to enter patient information via a GUI and the validation sub-system 22 may identify the patient information in the EMR system 26 based on the entered patient information.

The EMR system 26 may include a cloud server database and/or a database of a healthcare institution that stores patient information and/or the patient's electronic health records (e.g., patient records, medical records). The EMR system 26 may store a unique identifier for each patient, patient information, medical records of the patient, lab results of the patient, imaging reports of the patient, and so on. The EMR system 26 may associate the unique identifier and/or the patient information with the patient's electronic health medical records. For example, the EMR system 26 may store physiological parameters from the medical device 14 as part of the patient's electronic medical records. The clinician may access the patient's electronic medical records to determine a diagnosis for the patient, monitor a medical condition of the patient, view treatments provided to the patient. As such, it may be beneficial to store validated physiological parameters in the EMR system 26 to provide the clinician with accurate and/or up-to-date information for the diagnosis and/or monitoring.

The validation environment 10 may include the validation sub-system 22 that facilitates data transfer between the EMR system 26, the mobile device 18, and the medical device 14. For example, the validation sub-system 22 may be positioned within a cloud server and accessible via the network 24. The validation sub-system 22 may include a memory and a processor. The memory may be any suitable articles of manufacture that serves as media to store processor-executable code, data, or the like. These articles of manufacture may represent computer-readable media (e.g., any suitable form of memory or storage) that may store the processor-executable code used by the processor to perform the presently disclosed techniques. The memory may represent non-transitory computer-readable media (e.g., any suitable form of memory or storage) that may store the processor-executable code used by the processor to perform various techniques described herein. It should be noted that non-transitory merely indicates that the media is tangible and not a signal. The processor may be any type of computer processor or microprocessor capable of executing computer-executable code. As used herein, applications may include any suitable computer software or program that may be installed onto the validation sub-system 22 and executed by the processor. The processor may also include multiple processors that may perform the operations described below.

For example, the validation sub-system 22 may identify clinician information associated with the clinician performing the validation. For example, the clinician may log into an account via the application on the mobile device 18. The clinician may provide user credentials to access an account associated with the validation sub-system 22. The user credentials may include a username, a password, a pin, biometric data, a passphrase, and so on. If the user credentials match stored credentials in the validation sub-system 22, then the validation sub-system 22 may provide access to the user account. If the user credentials do not match the stored credentials, then the validation sub-system 22 may prompt the clinician to enter the user credentials. The user account may include clinician information such as a clinician name, a birth date, a clinician ID, and so on.

The validation sub-system 22 may identify the device information based on the device ID 16 and/or may identify the patient information based on the patient ID 20. The validation sub-system 22 may receive the device ID 16 from the mobile device 18. The validation sub-system 22 may identify the device information based on the device ID 16 in a database and/or the memory of the validation sub-system 22. The validation sub-system 22 may transmit an indication of the device information to the mobile device 18 for confirmation. The validation sub-system 22 may receive the patient ID 20 and identify patient information stored in an electronic medical record (EMR) system 26 based on the patient ID 20. The validation sub-system 22 may transmit the patient information to the mobile device 18. As such, the validation sub-system 22 may facilitate communication between the mobile device 18 and the EMR system 26.

In response to receiving two indications of validation, the validation sub-system 22 may associate the device ID 16 and the patient ID 20. For example, the validation sub-system 22 may receive a first indication of validation in response to transmitting the device information and a second indication of validation in response to transmitting the patient information. As such, the validation sub-system 22 may associate the physiological parameters monitored by the medical device 14 with the patient information in the EMR system 26. As such, the physiological parameters may be stored in the EMR system 26 with the patient's electronic medical records. In certain instances, the validation sub-system 22 may prompt the clinician for a third indication of validation to associate the device information with the patient information.

The validation sub-system 22 may receive an indication of validation from clinician via the mobile device 18. For example, the validation sub-system 22 may store physiological parameters from the medical device 14 during the patient monitoring session. After a period of time, the clinician may validate the physiological parameter based on a comparison between the physiological parameter displayed on the medical device 14 and the physiological parameter displayed on the mobile device 18 at a point in time. The validation sub-system 22 may transmit the stored physiological parameters that were generated by the medical device 14 over the period of time to the mobile device 18 for display. The physiological parameters may be displayed on the mobile device 18 and/or the medical device 14 as a waveform, a value, a status, and the like. The clinician may identify a portion of the physiological parameters without artifacts and/or representative of a physiological condition of the patient for validation. Additionally or alternatively, the clinician may compare the portion of the physiological parameter displayed on the mobile device 18 to an equivalent portion of the physiological parameter displayed on the medical device 14. If the physiological parameter displayed on the mobile device 18 matches the physiological parameter displayed on the medical device 14, then the clinician may provide an indication of validation. If the physiological parameter displayed on the mobile device 18 matches the physiological parameter displayed on the medical device 14, then the clinician may provide an indication of validation. In certain instances, the clinician may identify portions of the physiological parameter that may include artifacts, such as due to patient movement, and may not validate the portions via user input on the mobile device 18. In this way, the validation sub-system 22 may function as a clearinghouse for the physiological parameter.

The validation sub-system 22 may transmit the physiological parameters to the EMR system 26 with an indication of validation. For example, the validation sub-system 22 may transmit the physiological parameter at the point in time along with an indication of validation. The indication of validation may include a clinician ID associated with the clinician performing the validation, the patient ID 20, a time of the validation (e.g., the point in time), and the like. As such, a clinician using the validated physiological parameter for making a diagnosis and/or monitoring the patient may identify the clinician who recorded and validated the physiological parameter. Additionally, the validation sub-system 22 may transmit the stored physiological parameter to the EMR system 26 for storage. In other instances, the validation sub-system 22 may receive an indication of artifacts within the physiological parameter at the point in time and the validation sub-system 22 may transmit the physiological parameter at the point in time with the indication of artifacts for storage. As such, the validation sub-system 22 may provide an indication of accuracy and/or reliability of the physiological parameter.

In response to transmitting the validated physiological parameters to the EMR system 26, the validation sub-system 22 may transmit notification to the mobile device 18 for display. The notification may indicate a successful storage of the validated physiological parameters in the EMR system 26. In certain instances, the clinician may continue to monitor the physiological parameters of the patient over a second period of time of the patient monitoring session and return at a later time to validate the physiological parameters generated over the second period of time. In other instances, the clinician may view the notification and conclude the patient monitoring session. As such, the clinician may stop operation of the medical device 14, remove the one or more sensor(s) 12 from the patient, remove the medical device 14 and/or the one or more sensor(s) 12 from the validation environment 10, or any combination thereof.

It may be beneficial for the patient data stored in the EMR system 26 to include validated patient data (e.g., validated physiological parameters) to provide clinicians with accurate and/or up-to-date information and/or reduce risks of errors. For example, the clinician may spend less time verifying and/or correcting physiological parameters when using validated patient data in comparison to unvalidated patient data, thereby decreasing the amount of time to make a diagnosis and/or to monitor patient conditions. As such, the validation sub-system 22 may improve clinician workflow and/or reduce the amount of time for making the diagnosis and/or monitoring the patient condition.

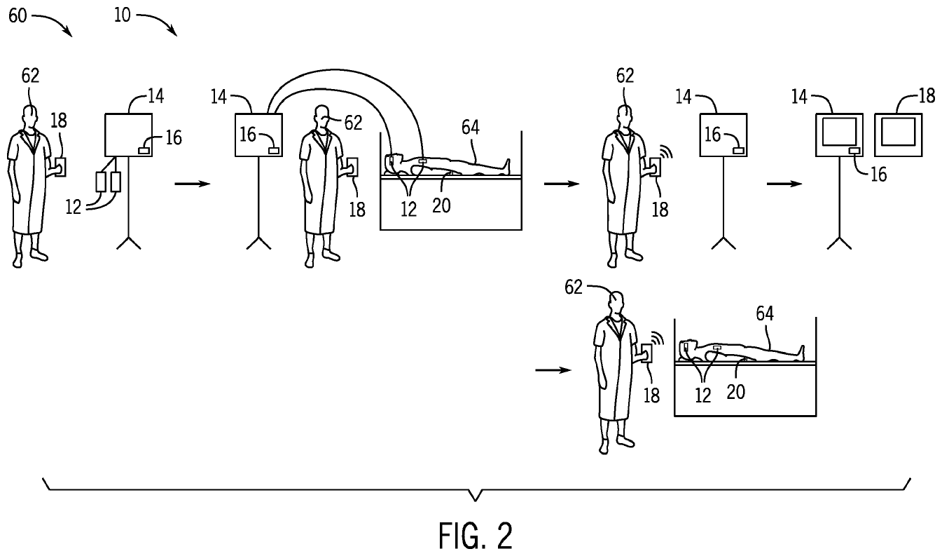

FIG. 2 is a workflow 60 of validating physiological parameters within the validation environment 10. At the start of a patient monitoring session, the clinician 62 may enter the validation environment 10. The environment may include the medical device 14 and one or more sensor(s) 12. As illustrated, the one or more sensor(s) 12 may be coupled to the medical device 14 via wires. In other embodiments, the one or more sensor(s) 12 may be wirelessly coupled to the medical device 14. The clinician 62 may also bring the mobile device 18 or other operator interface into the validation environment 10.

The clinician 62 may couple the one or more sensor(s) 12 to a patient 64 in the validation environment 10. For example, the clinician 62 may attach a sensor 12 to the patient 64 via and adhesive of the sensor 12. In another example, the clinician 62 may place a sensor 12 on a body part of the patient 64, such as a finger, the forehead, the chest, the arm, and so on. The one or more sensor(s) 12 may monitor one or more physiological parameter(s) of the patient 64 during the patient monitoring session. For example, the one or more sensor(s) 12 may transmit one or more signal(s) indicative of one or more physiological parameter(s) to the medical device 14 and the medical device 14 may determine the one or more physiological parameter(s) based on the one or more signal(s).

The clinician 62 may associate the medical device 14 with the patient by transmitting an indication of the device ID 16 and an indication of the patient ID to the validation sub-system 22 via the mobile device 18. For example, the clinician 62 may scan the device ID 16 of the medical device 14 using the mobile device 18 to provide an indication of the device ID 16 to the validation sub-system 22. The validation sub-system 22 may identify the medical device 14 in a list of stored and/or known patient medical devices 14 stored in a database and/or the memory of the validation sub-system 22 based on the device ID 16. The validation sub-system 22 may transmit the device information to the mobile device 18 to validate the device information. The clinician 62 may compare the displayed device information on the mobile device 18 with device information on the medical device 14 to validate the device information. The clinician 62 may scan the patient ID 20 on a bracelet worn by the patient 64 to provide an indication of the patient ID 20 to the validation sub-system 22. The validation sub-system 22 may identify patient information in the EMR system 26 based on the patient ID 20 and transmit the patient information to the mobile device 18 for validation. In response to an indication of validation of the device information and the patient information, the validation sub-system 22 may associate the device ID 16 and the patient ID 20. That is, the validation sub-system 22 may associate physiological parameters from the medical device 14 with the patient information and/or the unique identifier associated with the patient 64 in the EMR system 26.

After a period of time, the clinician 62 may validate one or more physiological parameter(s) of the patient 64. As discussed herein, the validation sub-system 22 may store one or more physiological parameter(s) from the patient-associated medical device 14 over the period of time. In an embodiment, the validation step that associates the device 14 with the particular patient may trigger a direct communication of the physiological parameter from the medical device 14 to both the validation sub-system 22 and the mobile device 18.

The validation sub-system 22 may transmit an indication of a physiological parameter to the mobile device 18 for display, for example in real-time. That is, the validation sub-system 22 may receive incoming physiological parameter data from the patient-associated medical device 14 that is (1) stored for possible inclusion in the EMR system 26 and also (2) passed, via the network 24, to the mobile device 18 in substantially real-time such that the real-time parameters displayed on the medical device 14 are also displayed on the mobile device 18. For certain medical devices 14, such as a blood pressure cuff, the calculated parameter may be stable after the cuff inflation/deflation cycle is complete. Thus, a blood pressure spot check may be completed, and the calculated blood pressure value displayed on the device 14 does not update until another cuff inflation event is triggered. For other types of monitoring, the rate of update of the physiological parameter may be such that communication of the physiological parameter to the mobile device 18 appears to be real-time even if there is a slight lag based on communication delays.

The clinician 62 may compare the physiological parameter displayed on the mobile device 18 to the physiological parameter displayed on the medical device 14. If the physiological parameter displayed on the mobile device 18 matches the physiological parameter displayed on the medical device 14, then the clinician 62 may provide a user input to validate the physiological parameters via the mobile device 18. In response to receiving an indication of the validation, the validation sub-system 22 may transmit the physiological parameter to the EMR system 26 along with the indication of validation for storage. In this way, the clinician 62 may spot check the physiological parameter. If the physiological parameter displayed on the mobile device 18 does not match the physiological parameter displayed on the medical device 14, then the clinician 62 may not validate the physiological parameter. In certain instances, the validation sub-system 22 may transmit the stored physiological parameters to the EMR system 26 without the indication of validation. In other instances, the validation sub-system 22 may delete the stored physiological parameters. Still in other instances, the validation sub-system 22 may continue the patient monitoring operations over a second time period and store physiological parameters from the medical device 14.

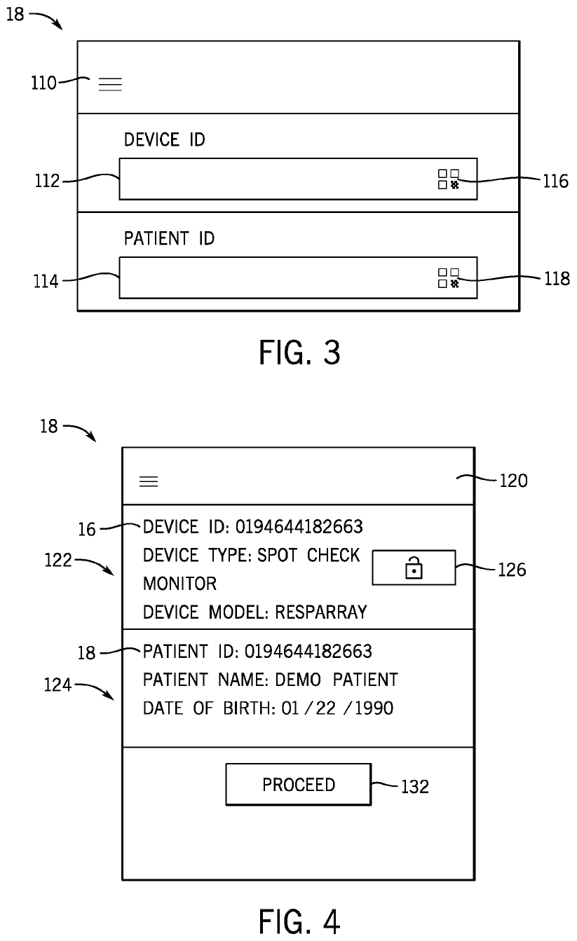

FIG. 3 is a schematic illustration of an example GUI 110 displayed by the mobile device 18 prior to receiving the device ID 16 and/or the patient ID 20 within the validation environment 10. The GUI 110 may include a first input box 112 to receive the device ID 16 and a second input box 114 to receive the patient ID 20. For example, the clinician may manually enter the device ID 16 into the first input box 112 using a keyboard. In response to receiving user selection of the first input box 112, the mobile device 18 may populate the GUI 110 with a keyboard to facilitate user entry of the device ID 16. Additionally or alternatively, the mobile device 18 may populate the GUI 110 with the keyboard in response to user selection of the second input box 114. In another example, the clinician may use the mobile device 18 to scan the device ID 16 and/or the patient ID 20 to enter them into the first input box 112 and the second input box 114, respectively. The GUI 110 may include a first icon 116 and/or a second icon 118 that activates a camera of the mobile device 18 in response to user selection. The camera may capture an indication of the device ID 16 and/or an indication of the patient ID 20 and transmit the device ID 16 and/or the patient ID 20 to the validation sub-system 22. For example, the camera may capture a barcode and/or a QR code indicative of the device ID 16 and/or the patient ID 20.

The validation sub-system 22 may retrieve information associated with the medical device 14 and/or the patient 64 in response to receiving the device ID 16 and/or the patient ID 20. For example, the validation sub-system 22 may retrieve device information associated with the medical device 14 from a database and/or a memory based on the device ID 16. In another example, the validation sub-system 22 may retrieve patient information 124 associated with the patient 64 from the EMR system 26 based on the patient ID 20. The validation sub-system 22 may populate a GUI with the device information 122 and the patient information in response to receiving the device ID 16 and the patient ID 20, respectively.

With the foregoing in mind, FIG. 4 is a schematic illustration of an example GUI 120 displayed by the mobile device 18 within the validation environment 10, where the GUI 120 includes information associated with the device ID 16 and/or the patient ID 20. The device information 122 may include the device ID 16, a type of device, a model of the device, and so on. The GUI 120 may also display the patient information 124 associated. As illustrated, the patient information 124 associated with the patient 64 may include a patient name, and/or a birth date. The patient information 124 may also include a gender of the patient 64, an admittance date of the patient 64, and so on.

The clinician may compare the information displayed on the GUI 120 to the device ID 16 positioned on the exterior surface of the medical device 14 and/or to the patient ID 20 on the bracelet worn by the patient 64. The clinician may select a second button 132 of the GUI 140 in response to determining that the displayed information matches the device ID 16 and/or the patient ID 20. By selecting the second button 132, clinician may validate an association between the medical device 14 and the patient 64. The mobile device 18 may transmit the association to the validation sub-system 22.

The GUI 140 may include a locking button 126 proximate to the device information that may associate the device ID 16 and/or the device information with the clinician in response to user selection. In a locked configuration, the GUI 120 may maintain an association between the device information and a clinician. As discussed herein, the clinician may provide authentication information when using the mobile device 18 for spot checks. For example, the medical device 14 associated with the device ID 16 may be set as a default device and the validation sub-system 22 may associate the medical device information with the clinician over a period of time. For example, the clinician may validate data generated by the medical device 14 and associated with a first patient 64, then use the medical device 14 to generate data associated with a second patient 64. The clinician may not provide the device ID 16 to the validation sub-system 22 between the first patient to the second patient. As such, the clinician may not repeatedly enter the device ID 16 when monitoring different patients. In this way, the clinician may perform fewer steps to validate the data in comparison to validating data without using the button locking 126. Rather, the clinician may enter the patient ID 20 to associate different patients with the medical device 14. After monitoring different patients and/or to associate a different medical device 14, the clinician may select the button 126 to transition the locking button 126 to an unlocked configuration. In the unlocked configuration, as illustrated, the GUI 120 may prompt for entry of the device ID 16.

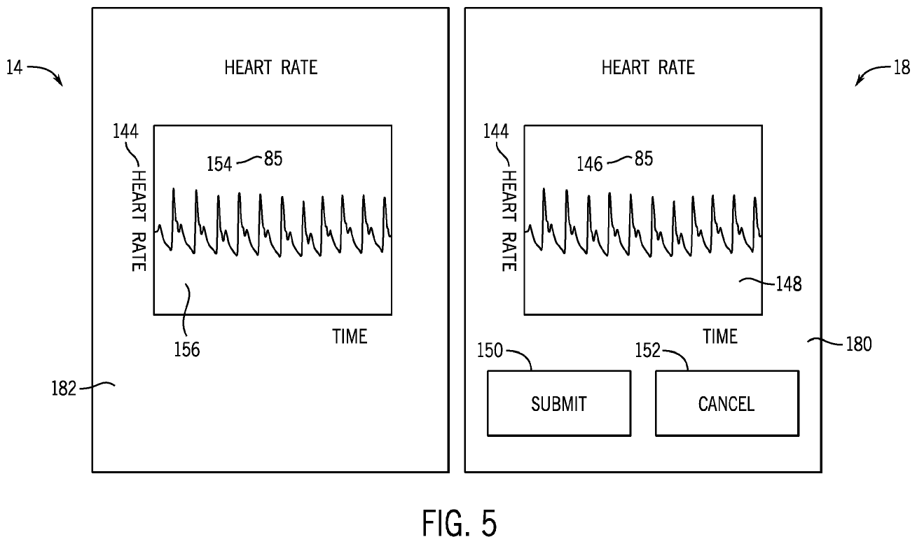

FIG. 5 is a schematic illustration of an example GUI 140 displayed by the mobile device 18 including a physiological parameter and an example GUI displayed by the medical device 14 including the physiological parameter over the same time period in the validation environment 10. In certain cases, the GUI may display a mirror of the GUI 142 displayed by the medical device 14 such that the displays are substantially identical. In other embodiments, the GUI 140 may be simplified such that only the numerical physiological parameter value or values from the device 14 is displayed.

The GUI 140 displays a portion of the physiological parameter 144 over a period of time (e.g., a most-recent time window). As illustrated, the physiological parameter 144 may include a heart rate, and GUI 140 may display a first indication 146 of the heart rate and a second indication 148 of the heart rate. The first indication 146 includes a value of the physiological parameter 144 at a point in time and the second indication 148 includes a graph with a waveform of the physiological parameter 144 over the period of time. The GUI 140 may also include a first button 150 indicative of validating the physiological parameter 144 and a second button 152 indicative of deleting the physiological parameter 144.

The GUI 142 displays a portion of the physiological parameter 144. As illustrated, the GUI 142 displays a first indication 154 of the physiological parameter 144 and a second indication 156 of the physiological parameter 144. The first indication 154 includes a value of the physiological parameter 144 at a point in time and the second indication 156 includes a graph with a waveform of the physiological parameter 144 over the period of time. The period of time and/or the point in time displayed by the GUI 140 may be the same period of time and/or the point of time displayed by the GUI 142. In certain instances, the clinician may select the period of time and/or the point of time by user selection on the mobile device 18 and/or the medical device 14. As such, the clinician may view the same or substantially similar physiological parameters 144 on the mobile device 18 and the medical device 14.

As discussed herein, the clinician 62 may validate the physiological parameter over the period of time and/or at the point in time if the physiological parameter 144 displayed by the mobile device 18 matches the physiological parameter 144 displayed by the medical device 14. The clinician 62 may indicate the validation by selecting the first button 150. The validation may be part of a spot check performed by the clinician. In response to selection of the first button 150, the mobile device 18 may transmit the physiological parameters to the validation sub-system 22 for storage in the EMR system 26. In response to receiving the user selection of the first button 150, the validation sub-system 22 may transmit the portion of the physiological parameter 144 over the period of time, the value of the physiological parameter 144 at the period of time, or both along with an indication of the validation to the EMR system 26. As such, the validation sub-system 22 may be a clearinghouse for validated physiological parameters. In response to selection of the second button, the mobile device 18 and/or the validation sub-system 22 may delete the physiological parameters from a memory and/or a database. In certain instances, the validation sub-system 22 may instruct the medical device 14 to generate updated physiological parameters and/or mobile device 18 to generate and/or display an additional GUI with the updated physiological parameters.

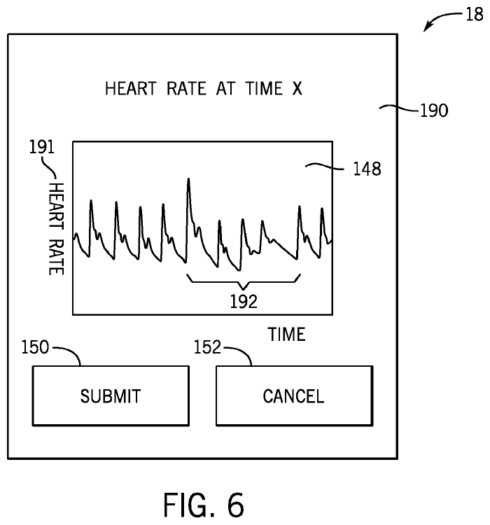

FIG. 6 is a schematic illustration of an example GUI 190 displayed by the mobile device 18 within the validation environment 10, where the GUI 190 displays a portion of the physiological parameter 191 over a period of time, e.g., a most-recent time window. The GUI 190 may include a second indication 148 of the physiological parameter 191. As illustrated, the physiological parameter 191 may include a heart rate, and the second indication 148 may include a waveform of the heart rate over the period of time. The portion of the physiological parameter 191 may include an artifact 192 over a subset of the period of time. The artifact 192 may include distortions to the waveform that may be caused by patient movement, transmission issues, electrical interference, and so on. For example, the artifact 192 may be a result of the patient 64 sneezing or by the patient 64 rolling over.

The clinician 62 may identify the artifact 192 and may not validate the physiological parameter 191. For example, the clinician 62 may select the second button 152 indicative of deleting the physiological parameter over the period of time. In other embodiments, the artifact 192 may be flagged by the device 14 such that the GUI 190 displays an indication of the artifact 192 (e.g., a change in color or other indication). In certain cases, a displayed physiological parameter value that is determined to be low confidence based on identified artifacts by the device 14 may be displayed in a different color on the GUI 190. In response to receiving user selection of the second button 152, the validation sub-system 22 may delete (e.g., purge) the stored physiological parameters from the database and/or the memory. That is, the validation sub-system 22 may not transmit the portion of physiological parameter 191 to the EMR system 26 for storage. In other embodiments, the validation sub-system 22 may transmit the stored physiological parameters to the EMR system 26 without the validation or with an indication of not validating the physiological parameter 144 at the point in time. As such, a clinician reviewing patient information in the EMR system 26 may understand that the physiological parameters 191 may not be as accurate and/or reliable as validated physiological parameters.

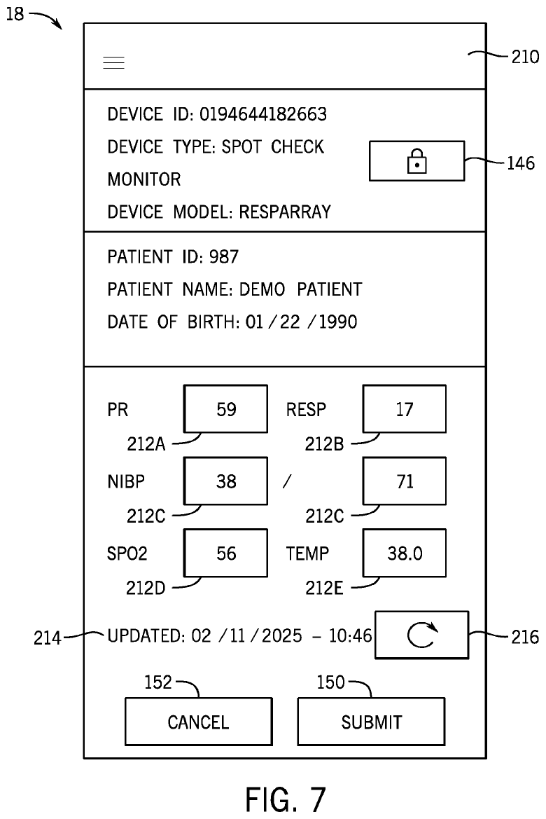

FIG. 7 is a schematic illustration of an example GUI 210 displayed by the mobile device 18 within the validation environment 10, where the GUI 210 displays physiological parameters generated by the medical device 14. The GUI 210 may include the physiological parameters 212 generated by the medical device 14. As illustrated, the GUI 210 may display a pulse rate 212A, a respiration rate 212B, a blood pressure 212C, a oxygen saturation 212D, and/or a temperature 212E. The clinician may compare the physiological parameters 212 to physiological parameters displayed on a display of the medical device 14. The clinician may select the first button 150 to validate the physiological parameters 212 of the GUI 210 based on a match between the physiological parameters 212 of the GUI 210 to the physiological parameters displayed on a display of the medical device 14. The physiological parameters 212 may be saved in the EMR system 26 with an indication of validation. For example, the indication of validation may include a time stamp 214 associated with the validation.

In certain instances, the GUI 190 may not be populated with all of the physiological parameters 212. For example, during start up of the medical device 14, the pulse rate 212A may be immediately monitored and populated into the GUI 190, but the blood pressure 212C may take a longer time to monitor. As such, the blood pressure 212C may not be populated into the GUI 210. After a period of time, the clinician may select a refresh button 216 to update the physiological parameters 212. During the period of time, the medical device 14 may generate the blood pressure, and as such, the GUI 210 may be updated to display the blood pressure 212C.

In other instances, as discussed herein, the clinician may identify an artifact within the physiological parameters 212. In other words, the physiological parameters 212 may not be representative of the clinical state of the patient 64. In response to identifying the artifact, the clinician may select the refresh button 216 to instruct the mobile device 18 to repopulate the GUI 210 with updated physiological parameters. The mobile device 18 may receive additional physiological parameters generated by the medical device 14 to repopulate the GUI 210. The clinician may perform an additional validation based on the additional physiological parameters.

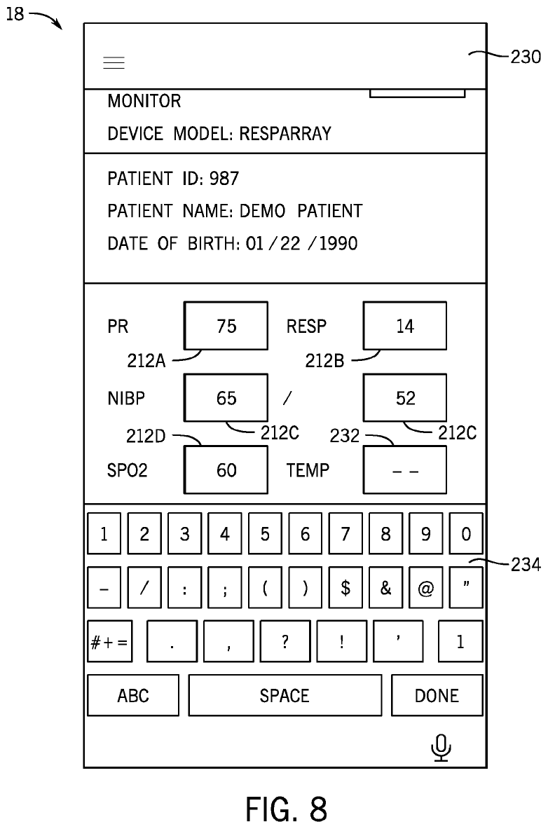

FIG. 8 is schematic illustration of an example GUI 230 displayed by the mobile device 18, where the GUI 230 displays physiological parameters 212 including a missing value. In certain instances, the medical device 14 may not be equipped to monitor and/or generate a physiological parameter 212. For example, the medical device 14 may not include capabilities to measure the physiological parameter. As such, an indication of a missing value 232 be displayed on the GUI 230.

In certain instances, the clinician may manually enter the physiological parameter 212 via a keyboard 234. In response to user selection of the indication of the missing value 232, the mobile device 18 may populate the GUI 230 with a keyboard 234. The clinician may enter the physiological parameter 212 using the keyboard 234. The clinician may acquire the physiological parameter via an additional medical device 14 that may not be communicatively coupled to the mobile device 18. For example, the clinician may use a thermostat to acquire the temperature of the patient 64 and use the keyboard 234 to enter the temperature value into the GUI 230.

In other instances, the clinician may submit the physiological parameters 212 with the missing value. Still in other instances, the clinician may remove physiological parameters 212 that may not be representative of the clinical state of the patient 64. For example, the clinician may view the patient 64 sneezing during a blood pressure measurement and determine that the measured blood pressure may not be reliable. The clinician may use the refresh button (e.g., the refresh button 216 of FIG. 7) to cause the medical device 14 to update the physiological parameters. In another example, the clinician may use a blood pressure monitoring to determine the blood pressure of the patient 64 and manually enter the blood pressure by selecting input boxes associated with the blood pressure 212C. As such, the clinician may ensure that the physiological parameters 212 transmitted to the EMR system 26 are accurate representations of the clinical state of the patient 64.

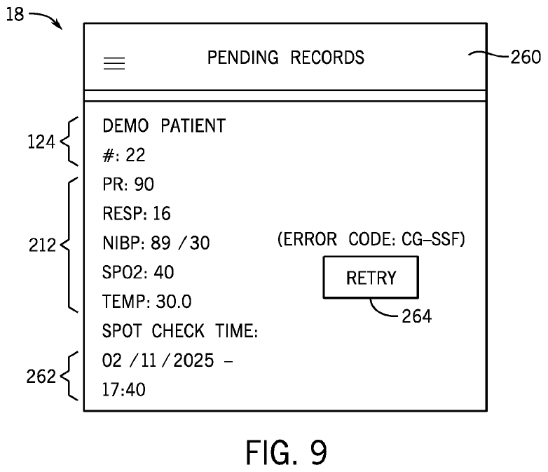

FIG. 9 is a schematic illustration of an example GUI 260 displayed by the mobile device 18 in the validation environment 10, where the GUI 260 displays pending records (e.g., physiological parameters 212) for storage in the EMR system 26 at a subsequent time different from the acquisition and/or validation time. For example, the mobile device 18 may enter a location without a connection to the network 24 or with poor connection to the network 24. For example, the mobile device 18 may determine if a signal strength of the connection may be below a threshold signal strength. The threshold signal strength may be a signal strength used to transmit data within the validation environment 10. If the signal strength is below the threshold signal strength, the mobile device 18 may store the physiological parameters 212 in the memory to transmit at a subsequent time, such as when the signal strength may be above the threshold signal strength. As such, the mobile device 18 may not be able to transmit the validated physiological parameters to the validation sub-system 22 and/or the EMR system 26. The mobile device 18 may temporarily store the physiological parameters in a memory.

The GUI 260 may patient information 124, associated physiological parameters 212, and/or an indication of validation 262. The indication of validation 262 may include a date and/or a time associated with the clinician validating the physiological parameters 212. As such, the clinician may view the GUI 260 to identify a number of physiological parameters 212 for submission to the EMR system 26.

The GUI 260 may also include a retry button 264 that prompts the mobile device 18 to transmit the physiological parameters 212 to the validation sub-system 22. In response user selection of the retry button, the mobile device 18 may attempt to transmit the physiological parameters 212 to the validation sub-system 22. The mobile device 18 may successfully transmit the physiological parameters 212 if the mobile device 18 is connected to the network 24. In certain instances, the mobile device 18 may not be connected to the network 24 and may not transmit the physiological parameters 212. As such, the mobile device 18 may populate the GUI 260 with an error message to indicate that the physiological parameters 212 may not be transmitted.

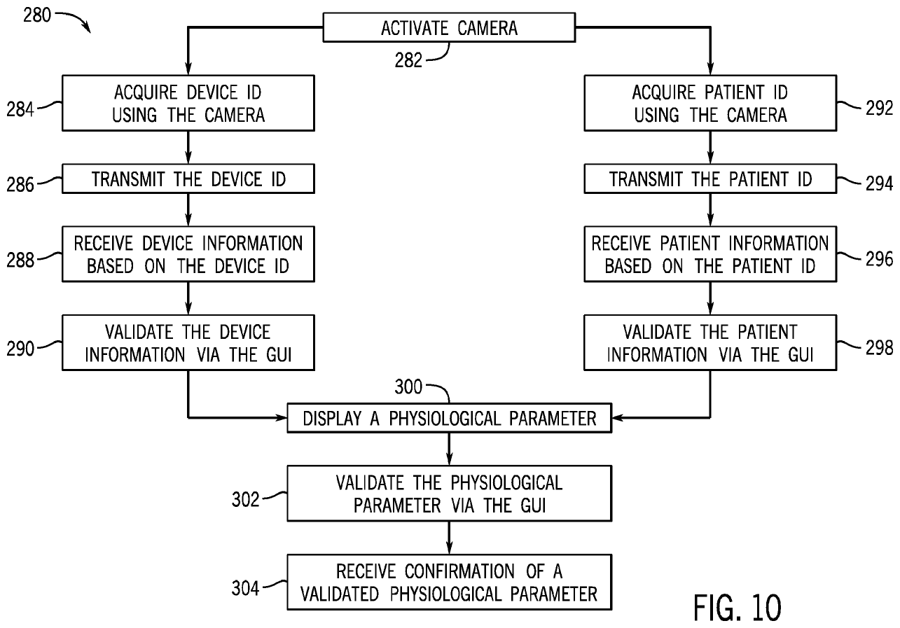

FIG. 10 is a flow diagram of an example method 280 for validating the physiological parameters using the mobile device 18 in the validation environment 10. As discussed herein, the mobile device 18 may facilitate an association between the physiological parameters from the medical device 14 and the patient information in the EMR system 26.

At block 282, the mobile device 18 may activate a camera. For example, the clinician 62 may activate an application on the mobile device 18, which may cause the camera to activate. In other instances, the clinician 62 may activate the camera on the mobile device 18 to scan the device 1D 16 and/or the patient ID 20.

At block 284, the mobile device 18 may acquire a device ID 16 using the camera. The clinician 62 may use the mobile device 18 to scan the device ID 16 of the medical device 14. The mobile device 18 may receive a barcode and/or a QR code indicative of the device ID 16.

At block 286, the mobile device 18 may transmit the device ID 16. The mobile device 18 may transmit the device ID 16 to the validation sub-system 22. The validation sub-system 22 may compare the device ID 16 to stored device information to identify device information corresponding to the medical device 14.

At block 288, the mobile device 18 may receive the device information based on the device ID 16. The mobile device 18 may receive the device information from the validation sub-system 22 and display the device information via a GUI on the display. The device information may include a make and/or model, a functionality, a name, and so on of the medical device 14.

At block 290, the mobile device 18 may validate the device information via the GUI. The mobile device 18 may receive user selection of the first button of the GUI indicative of validating the device information. For example, the clinician 62 may compare the device information displayed on the mobile device 18 with device information on the medical device 14 to validate the device information.

At block 292, the mobile device 18 may acquire patient ID using the camera. The clinician 62 may use the mobile device 18 to scan the patient ID 20 on a bracelet worn by the patient 64. The mobile device 18 may receive a barcode and/or a QR code indicative of the patient ID 20.

At block 294, the mobile device 18 may transmit the patient ID 20. The mobile device 18 may transmit the patient ID 20 to the validation sub-system 22. The validation sub-system 22 may identify patient information in the EMR system 26 based on the patient ID 20. The validation sub-system 22 may transmit the patient information to the mobile device 18 for display.

At block 296, the mobile device 18 may receive patient information based on the patient ID 20. The mobile device 18 may receive the patient information from the validation sub-system 22 and display the patient information via a GUI on the display. The patient information may include a patient name, a birth date, an address, and the like.

At block 298, the mobile device 18 may validate the patient information via the GUI. For example, the clinician 62 may compare the patient information displayed on the mobile device 18 with patient information on the bracelet worn by the patient 64. The clinician 62 may provide user selection of a first button on the GUI indicative of validating the patient information. The mobile device 18 may receive an indication of confirming and/or validating the patient information via the first button on the GUI. The mobile device 18 may transmit an indication of the confirmation and/or validation to the validation sub-system 22.

At block 300, the mobile device 18 may display a physiological parameter. The mobile device 18 may display a GUI with an indication of the physiological parameter over a period of time and/or at a point in time. For example, the indication may include a waveform of the physiological parameter, a value of the physiological parameter, or both. In another example, the indication may include a status of the physiological parameter, such as a status of an impaired state of autoregulation or an intact state of autoregulation.

At block 302, the mobile device 18 may validate for the physiological parameter via the GUI. For example, the mobile device 18 may receive user selection of a first button of the GUI indicative of validating the physiological parameter. The mobile device 18 may transmit the user selection of the first button to the validation sub-system 22 to transmit the portion of the physiological parameter to the EMR system 26 along with an indication of validation. As such, the validation sub-system 22 may transmit a snapshot of a validated physiological parameter.

At block 304, the mobile device 18 may receive confirmation of a validated physiological parameter. The mobile device 18 may display a notification indicative of a successful transfer of the physiological parameter and the indication of validation from the validation sub-system 22 to the EMR system 26. The notification may include the portion of the physiological parameter, the clinician ID, a time stamp, the patient ID 20, and so on.

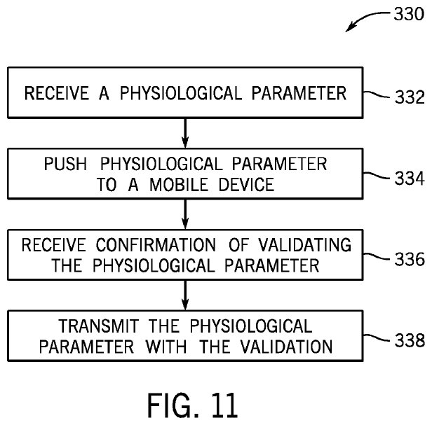

FIG. 11 is a flow diagram of an example method 330 for validating and storing the physiological parameter in the EMR system 26 in the environment of FIG. 1. The clinician 62 may periodically perform a spot check on the physiological parameters and validate physiological parameters, thereby reducing and/or eliminating validation of physiological parameters stored in the EMR system 26 and simplifying workflows for making a diagnosis and/or monitoring patient conditions. For example, the clinician may perform a spot check once an hour, once every other hour, and so on. As discussed herein, the validation sub-system 22 may function as a clearinghouse for validated physiological parameters. The validation sub-system 22 may store physiological parameters for a period of time (e.g., during the patient monitoring session), and in response to validating the physiological parameters, the validation sub-system 22 may transmit the physiological parameters for storage in the EMR system 26 along with an indication of the validation. By way of specific example, the medical device 14 may include a laryngoscope that generates image data of the patient.

At block 332, the validation sub-system 22 may receive a physiological parameter. For example, the validation sub-system 22 may receive image data and/or video data from a laryngoscope (e.g., medical device 14), imaging settings of the laryngoscope, or both. The image data and/or video data may include a patient being intubated in the validation environment 10. The validation sub-system 22 may receive and store the physiological parameter from the medical device 14 over a period of time.

At block 334, the validation sub-system 22 may push the physiological parameter to a mobile device 18. The validation sub-system 22 may instruct the mobile device 18 to display a GUI with an indication of the physiological parameter to the mobile device 18. For example, the GUI may display one or more thumbnails that correspond to video data and/or image data generated by the laryngoscope. The GUI may display a video and/or an image in response to user selection of a thumbnail. In certain instances, the clinician may validate the intubation, such as whether the intubation may be good (e.g., a successful placement of the laryngoscope into the patient's mouth, nose, or throat) or bad (e.g., an unsuccessful placement of the laryngoscope). The GUI may also include a first button indicative of validating the physiological parameter and a second button indicative of deleting the physiological parameter. For example, the image data and/or video data from the laryngoscope may be validated via the GUI during a spot check by the clinician.

At block 336, the validation sub-system 22 may receive confirmation of validating the physiological parameter. For example, the mobile device 18 may receive user selection of the first button on the GUI indicative of validating the stored physiological parameter. In certain instances, validating the stored physiological parameter may include indicating that placement of the laryngoscope is good. The validation sub-system 22 may store the association with the video and/or image selected in block 284. The validation sub-system 22 may receive the confirmation via the mobile device 18.

At block 338, the validation sub-system 22 may transmit the physiological parameter with the validation. For example, the validation sub-system 22 may transmit the physiological parameter including the imaging settings of the laryngoscope, the video and/or image generated by the laryngoscope, and/or the indication of the validation (e.g., good placement, bad placement) for storage in the EMR system 26. The indication of validation may also include with a time stamp associated with the validation, a clinician ID that performed the validation, the patient ID, a portion of the physiological parameter, and so on. As such, the physiological parameter transmitted to the EMR system 26 may include validated physiological parameters, thereby reducing the step of validated stored physiological parameters in the EMR system 26. The clinician may use the validated physiological parameters to form a diagnosis for the patient and/or to monitor patient conditions, which may improve patient care by providing accurate and/or up-to-date information.

The methods discussed herein include various steps represented by blocks in flow diagrams. It should be noted that at least some steps may be performed as an automated procedure by one or more components of a system. Although the flow diagrams may illustrate the steps in a certain sequence, it should be understood that the steps may be performed in any suitable order and certain steps may be carried out simultaneously, where appropriate. Additionally, steps may be added to or omitted from the methods.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

The invention may be further described by reference to the following examples:

Example 1: A system including a medical device to monitor a parameter of a patient or administer a treatment to the patient, the medical device including a device identifier and a validation system communicatively coupled to the medical device, the validation system including a processor and a memory a memory storing instructions that, when executed by the processor, cause the processor to perform operations to cause a mobile device to receive the device identifier and a patient identifier, display the device identifier and the patient identifier on a graphical user interface (GUI) displayed on the mobile device, receive user input associating the medical device with the patient based on user input from the GUI, wherein the patient is associated with the patient identifier, send a validated association based on the user input, and instruct the mobile device to display the parameter responsive to receiving the validated association.

Example 2: The system of example 1, wherein the instructions, when executed by the processor, cause the processor to perform operations to receive clinician credentials associated with a clinician identifier from the mobile device prior to receiving the device identifier from the mobile device and validate the clinician identifier based on stored clinician credentials.

Example 3: The system of example 2, wherein the instructions, when executed by the processor, cause the processor to associate the medical device with the clinician identifier over a period of time in response to user selection of a first button and block the mobile device from receiving an additional device identifier.

Example 4: The system of example 1, wherein the instructions, when executed by the processor, cause the processor to cause display of a parameter validation via the GUI on the mobile device and receive a second indication of validation of the parameter via the GUI.

Example 5: The system of example 4, wherein the GUI includes a representation of the parameter as a numerical value, a first button indicative of validating the parameter, and a second button indicative of not validating the parameter and wherein the second indication includes user selection of the first button.

Example 6: The system of example 1, wherein the medical device includes a display to display the parameter.

Example 7: The system of example 6, wherein the parameter displayed on the medical device is displayed concurrently on the mobile device.

Example 8: The system of example 1, wherein the instructions, when executed by the processor, cause the processor to instruct the mobile device to store the parameter in a memory based on a signal strength between the mobile device and a network being below a threshold and display the parameter in a list of pending records on the GUI displayed on the mobile device.

Example 9: The system of example 8, wherein the instructions, when executed by the processor, cause the processor to after a period of time, receive a second indication from the mobile device indicative of transmitting the parameter to an electronic medical record system and transmit the parameter to the electronic medical record system based on the signal strength between the mobile device and the network being above the threshold.

Example 10: A system including a medical device to monitor a first parameter of a patient or a second parameter of the medical device, the medical device including a device identifier, a mobile device to acquire a patient identifier and the device identifier, and a validation system coupled to the medical device and the mobile device, the validation system including a processor and a memory storing instructions that, when executed by the processor, cause the processor to receive the first parameter or the second parameter from the medical device, instruct the mobile device to display the first parameter or the second parameter, receive a first user input indicative of validating the first parameter or the second parameter from the mobile device at a point in time, and transmit the validated first parameter or the validated second parameter to an electronic medical record system with an indication of validation.

Example 11: The system of example 10, wherein the instructions, when executed by the processor, cause the processor to receive the device identifier and the patient identifier from the mobile device, wherein the instructions to transmit the validated first parameter or the validated second parameter include instructions to store the validated first parameter or the validated second parameter in an electronic medical record of the patient associated with the patient identifier.

Example 12: The system of example 11, wherein the instructions, when executed by the processor, cause the processor to access the electronic medical record system to identify patient information of the patient based on the patient identifier.

Example 13: The system of example 10, wherein the instructions, when executed by the processor, cause the processor to generate the indication of the validation based on a time of the validation, a clinician identifier associated with a clinician performing the validation, the patient identifier, or any combination thereof.

Example 14: The system of example 10, wherein the instructions, when executed by the processor, cause the processor to instruct the mobile device to display the first parameter or the second parameter over a second period of time, receive a second user input indicative of not validating the first parameter or the second parameter from the mobile device at a second point in time, and transmit the first parameter or the second parameter to the electronic medical record system without the indication of the validation.

Example 15: A system including a medical device to monitor a patient or administer a treatment to the patient, wherein the medical device outputs a parameter indicative of a physiological parameter of the patient, a validation system communicatively coupled to the medical device and an electronic medical record system, the validation system receives the parameter and transmits the parameter to the electronic medical record system, and a mobile device communicatively coupled to the validation system, the mobile device operating a software application programmed with instructions to activate a camera, acquire, via the camera, a device identifier of the medical device, receive device information associated with the medical device in response to transmitting the device identifier to the validation system, acquire, via the camera, a patient identifier of the patient, receive patient information associated with the patient in response to transmitting the patient identifier to the validation system, validate the device information and the patient information in response to receiving a first user selection from the GUI, validate the parameter in response to receiving a second user selection from the GUI displaying the parameter, and display a confirmation of transmitting the parameter to the electronic medical record system with an indication of validation.

Example 16: The system of example 15, wherein the software application is programmed with instructions to display the physiological parameter as a waveform, a numerical value, or both.

Example 17: The system of example 15, wherein the software application is programmed with instructions to display the device information for validation, wherein the device information includes a make of the medical device, a model of the medical device, a functionality of the medical device, a name of the medical device, or any combination thereof.

Example 18: The system of example 15, wherein the software application is programmed with instructions to display the patient information for validation, wherein the patient information includes a patient name, a birth date, an address, a gender, a phone number, or any combination thereof.

Example 19: The system of example 15, wherein the software application is programmed with instructions to receive clinician credentials associated with a clinician identifier prior to acquiring the device identifier.

Example 20: The system of example 19, wherein the software application is programmed with instructions to display the confirmation with a time stamp of the validation, the clinician identifier, the patient identifier, or any combination thereof.

1. A system comprising:
a medical device to monitor a parameter of a patient or administer a treatment to the patient, the medical device comprising a device identifier; and
a validation system communicatively coupled to the medical device, the validation system comprising:
a processor; and
a memory storing instructions that, when executed by the processor, cause the processor to perform operations to:
cause a mobile device to receive the device identifier and a patient identifier;
display the device identifier and the patient identifier on a graphical user interface (GUI) displayed on the mobile device;
receive user input associating the medical device with the patient based on user input from the GUI, wherein the patient is associated with the patient identifier;
send a validated association based on the user input; and
instruct the mobile device to display the parameter responsive to receiving the validated association.

2. The system of claim 1, wherein the instructions, when executed by the processor, cause the processor to perform operations to:
receive clinician credentials associated with a clinician identifier from the mobile device prior to receiving the device identifier from the mobile device; and
validate the clinician identifier based on stored clinician credentials.

3. The system of claim 2, wherein the instructions, when executed by the processor, cause the processor to:
associate the medical device with the clinician identifier over a period of time in response to user selection of a first button; and
block the mobile device from receiving an additional device identifier.

4. The system of claim 1, wherein the instructions, when executed by the processor, cause the processor to perform operations to:
cause display of a parameter validation via the GUI on the mobile device; and
receive a second indication of validation of the parameter via the GUI.

5. The system of claim 4, wherein the GUI comprises a representation of the parameter as a numerical value, a first button indicative of validating the parameter, and a second button indicative of not validating the parameter and wherein the second indication comprises user selection of the first button.

6. The system of claim 1, wherein the medical device comprises a display to display the parameter.

7. The system of claim 6, wherein the parameter displayed on the medical device is displayed concurrently on the mobile device.

8. The system of claim 1, wherein the instructions, when executed by the processor, cause the processor to:
instruct the mobile device to store the parameter in a memory based on a signal strength between the mobile device and a network being below a threshold; and
display the parameter in a list of pending records on the GUI displayed on the mobile device.

9. The system of claim 8, wherein the instructions, when executed by the processor, cause the processor to:
after a period of time, receive a second indication from the mobile device indicative of transmitting the parameter to an electronic medical record system; and
transmit the parameter to the electronic medical record system based on the signal strength between the mobile device and the network being above the threshold.

10. A system comprising:
a medical device to monitor a first parameter of a patient or a second parameter of the medical device, the medical device comprising a device identifier;
a mobile device to acquire a patient identifier and the device identifier; and
a validation system coupled to the medical device and the mobile device, the validation system comprising:
a processor; and
a memory storing instructions that, when executed by the processor, cause the processor to:
receive the first parameter or the second parameter from the medical device;
instruct the mobile device to display the first parameter or the second parameter;
receive a first user input indicative of validating the first parameter or the second parameter from the mobile device at a point in time; and
transmit the validated first parameter or the validated second parameter to an electronic medical record system with an indication of validation.

11. The system of claim 10, wherein the instructions, when executed by the processor, cause the processor to:
receive the device identifier and the patient identifier from the mobile device, wherein the instructions to transmit the validated first parameter or the validated second parameter comprise instructions to store the validated first parameter or the validated second parameter in an electronic medical record of the patient associated with the patient identifier.

12. The system of claim 11, wherein the instructions, when executed by the processor, cause the processor to access the electronic medical record system to identify patient information of the patient based on the patient identifier.

13. The system of claim 10, wherein the instructions, when executed by the processor, cause the processor to generate the indication of the validation based on a time of the validation, a clinician identifier associated with a clinician performing the validation, the patient identifier, or any combination thereof.

14. The system of claim 10, wherein the instructions, when executed by the processor, cause the processor to:
instruct the mobile device to display the first parameter or the second parameter over a second period of time;
receive a second user input indicative of not validating the first parameter or the second parameter from the mobile device at a second point in time; and
transmit the first parameter or the second parameter to the electronic medical record system without the indication of the validation.

15. A system comprising:
a medical device to monitor a patient or administer a treatment to the patient, wherein the medical device outputs a parameter indicative of a physiological parameter of the patient;
a validation system communicatively coupled to the medical device and an electronic medical record system, the validation system receives the parameter and transmits the parameter to the electronic medical record system; and
a mobile device communicatively coupled to the validation system, the mobile device operating a software application programmed with instructions to:
activate a camera;
acquire, via the camera, a device identifier of the medical device;
receive device information associated with the medical device in response to transmitting the device identifier to the validation system;
acquire, via the camera, a patient identifier of the patient;
receive patient information associated with the patient in response to transmitting the patient identifier to the validation system;
validate the device information and the patient information in response to receiving a first user selection from the GUI;
validate the parameter in response to receiving a second user selection from the GUI displaying the parameter; and
display a confirmation of transmitting the parameter to the electronic medical record system with an indication of validation.

16. The system of claim 15, wherein the software application is programmed with instructions to display the physiological parameter as a waveform, a numerical value, or both.

17. The system of claim 15, wherein the software application is programmed with instructions to display the device information for validation, wherein the device information comprises a make of the medical device, a model of the medical device, a functionality of the medical device, a name of the medical device, or any combination thereof.

18. The system of claim 15, wherein the software application is programmed with instructions to display the patient information for validation, wherein the patient information comprises a patient name, a birth date, an address, a gender, a phone number, or any combination thereof.

19. The system of claim 15, wherein the software application is programmed with instructions to receive clinician credentials associated with a clinician identifier prior to acquiring the device identifier.

20. The system of claim 19, wherein the software application is programmed with instructions to display the confirmation with a time stamp of the validation, the clinician identifier, the patient identifier, or any combination thereof.
